(19) United States
(12) Patent Application Publication
(10) Pub. No.: US 2014/0081155 A1
(43) Pub. Date: Mar. 20, 2014

(54) Methods And Apparatus for Wireless Electrode Having Power Conservation

(71) Applicant: Scott R. Coggins, Littleton, MA (US)

(72) Inventor: Scott R. Coggins, Littleton, MA (US)

(21) Appl. No.: 13/616,594

(22) Filed: Sep. 14, 2012

(51) Int. Cl. A61B 5/00; A61B 5/044; A61B 5/01; A61B 5/0408; A61B 5/0205

(52) U.S. Cl. CPC A61B 5/0006; A61B 5/0408; A61B 5/02055; A61B 5/01; A61B 5/0008; A61B 5/044; A61B 5/746; A61B 5/021
USPC 600/483; 600/509; 600/485; 600/523

(57) ABSTRACT

Methods and apparatus provide a wireless electrode having energy conservation. In one embodiment, a wireless electrode includes a radio module coupled to the sensor to wirelessly transmit the cardiac information, an energy source to power the radio module, and an activation mechanism coupled to the energy source, the activation mechanism having an activated state in which power from the energy source is delivered to the radio module and a non-activated state in which power from the energy source is not delivered to the radio module.

### FIELD

The present invention relates generally to medical electrodes, more particularly, to a wireless medical electrode.

### BACKGROUND

As is known in the art, a wide variety of sensors for obtaining physiological parameters are available. For example, electrocardiograph (ECG) systems include sensors for detecting cardiac information from a patient. A conventional ECG system includes a series of patch electrodes for attachment to the chest and other locations. The patch electrodes are coupled to a base system, which typically includes a display monitor to allow medical personnel to monitor the patient heartbeat pattern, pulse rate etc. It will be appreciated that the number of wires extending from the patient to the monitor can be significant. In addition, if other medical equipment is connected to the patient, it can be challenging to maintain the correct connections to the patient, particularly for uncooperative patients. Further, in the event that a patient must be moved quickly due to a medical emergency, connections to medical equipment can be problematic.

To address excessive mechanical connections to a patient, wireless systems have been developed. For example, wireless ECG systems typically include electrodes extending from a patient's skin to a base system secured to the patient's bed. The base system wirelessly transmits sensor information to a remote monitor, which can be secured to the wall of a hospital room and/or nursing station. However, this arrangement still requires significant mechanical connections from the patient to a base system.

Wireless electrodes have been developed to provide self-contained sensors that are attachable to a patient. The wireless electrodes wirelessly transmit physiological information to a remote monitor. While wireless electrodes eliminate the need for mechanical connections to a patient, the electrodes have certain limitations, such as battery life. It will be appreciated that battery life is at a premium. In conventional wireless electrodes, the battery may transmit information even when the electrode is not connected to a patient. This can result in a reduction in the useful life of a wireless electrode by wasting battery power. In addition, it may result in a sensor not monitoring patent information due to a depleted battery, which can have disastrous results. Also, wasting battery power results in medical personnel spending more time to replace and/or recharge batteries, time which is then not spent directly for patient care.

### SUMMARY

The present invention provides method and apparatus for a wireless electrode that transmits only when a transmitter is attached to a patient. In exemplary embodiments, a radio module can be coupled to the electrode to activate the device. While illustrative embodiments are shown having certain configurations, structures, and applications, it is understood that the invention is applicable to electrodes in general for which it is desirable to conserve power.

In one aspect of the invention, a wireless electrode comprises an interface surface to contact a patient, a sensor coupled to the interface surface to detect patient cardiac information, a radio module coupled to the sensor to wirelessly transmit the cardiac information, an energy source to power the radio module, and an activation mechanism coupled to the energy source, the activation mechanism having an activated state in which power from the energy source is delivered to the radio module and a non-activated state in which power from the energy source is not delivered to the radio module.

The electrode can further include one or more of the following features: the activation mechanism requires manual manipulation to transition to the activated state, the activation mechanism comprises a deformable receptacle to receive a manually insertable structure for transition to the activated state, the radio module comprises a transmitter insertable into the electrode, insertion of the transmitter into the electrode transitions the activation mechanism to the activated state, a temperature sensor, the radio module is disabled when the temperature sensor does not sense a temperature greater than a threshold, the radio module transmits information from the temperature sensor, the energy source comprises a battery, the energy source further comprises a photovoltaic device coupled to the battery, and/or the electrode comprises an ECG electrode.

In another aspect of the invention, a system to obtain patient information comprises: a plurality of wireless electrodes, each comprising: an interface surface to contact a patient, a sensor coupled to the interface surface to detect patient cardiac information, a radio module coupled to the sensor to wirelessly transmit the cardiac information, an energy source to power the radio module, and an activation mechanism coupled to the energy source, the activation mechanism having an activated state in which power from the energy source is delivered to the radio module and a non-activated state in which power from the energy source is not delivered to the radio module, and a transmit/receive module to receive the cardiac information from the radio module, and a monitor in communication with the transmit/receive module to display the cardiac information and generate alerts based upon the cardiac information.

The system can further include one or more of the following features: at least one of the wireless electrodes includes a temperature sensor, the radio module is disabled when the temperature sensor does not sense a temperature greater than a threshold, the energy source comprises a photovoltaic mechanism coupled to a battery, and/or the activation mechanism cannot transition to the activated state unless the radio module is present, wherein the radio module comprises a transmitter insertable into the electrode, and insertion of the transmitter into the electrode transitions the activation mechanism to the activated state.

In a further aspect of the invention, a method of providing a wireless electrode comprises: providing an interface surface to contact a patient, providing a sensor coupled to the interface surface to detect patient cardiac information, providing a radio module coupled to the sensor, the radio module including a transmitter to wirelessly transmit the cardiac information, providing an energy source to power the radio module, and providing an activation mechanism coupled to the energy source, the activation mechanism having an activated state in which power from the energy source is delivered to the radio module and a non-activated state in which power from the energy source is not delivered to the radio module.

The method can further include providing a temperature sensor, wherein the radio module is disabled when the temperature sensor does not sense a temperature greater than a threshold, and/or insertion of the transmitter into the electrode transitions the activation mechanism to the activated state.

In a further aspect of the invention, a method comprises: applying a wireless electrode to a patient to obtain patient cardiac information, manipulating an activation mechanism of the wireless electrode from a non-activated state in which power from an energy source is not delivered to a radio module to an activated state in which power from the energy source is delivered to the radio module, and monitoring the cardiac information transmitted by the radio module.

### BRIEF DESCRIPTION OF THE DRAWINGS

The foregoing features of this invention, as well as the invention itself, may be more fully understood from the following description of the drawings in which:

FIG. 1 is a schematic representation of a wireless electrode having power conservation in accordance with exemplary embodiments of the invention;

FIG. 2 is functional block diagram of a wireless electrode having power conservation in accordance with exemplary embodiments of the invention;

FIG. 3A shows a wireless electrode and a radio module in a non-engaged first position and FIG. 3B shows the wireless electrode and the radio module in an engaged second position;

FIG. 4 shows a schematic representation of an exemplary activation mechanism having a first portion on the electrode and a second portion on the radio module; and

FIG. 5 is a flow diagram showing exemplary steps to activate a wireless electrode.

### DETAILED DESCRIPTION

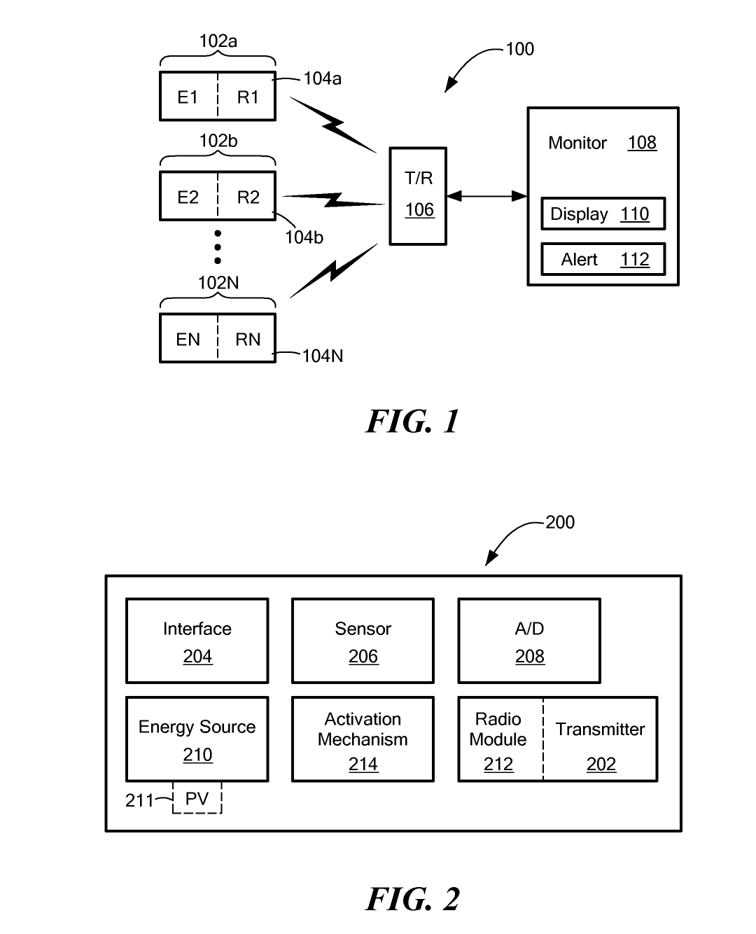

FIG. 1 shows an exemplary patient monitoring system 100 having wireless electrodes 102a-N with radio modules 104a-N, or portions thereof, that can be selectively attached to the electrodes. The wireless electrodes 102 can monitor patient information, such as cardiac information, as part of a wireless ECG (electrocardiograph) system. In other embodiments, the electrodes monitor blood pressure, temperature and/or other physiological information.

The patient monitoring system 100 includes a transmit/receive module 106 to wirelessly receive information from the electrodes 102. The transmit/receive module 106 can be wirelessly or mechanically connected to a monitor module 108, which can include a display 110 to enable medical personnel to view the patent heartbeat, for example. The monitor module 108 can further include an alert module 112 to generate an alert in the event of cardiac arrest or other cardiac stress condition. It is understood that the processing of patient cardiac data and alert generation are well known in the art.

FIG. 2 shows an exemplary wireless electrode 200 with a discrete transmit module 202 that can be engaged with the electrode to activate the device in accordance with exemplary embodiments of the invention. The wireless electrode 200 includes an interface 204 for electro-mechanical contact with the skin of a patient. A sensor 206 is coupled to the interface 204 to receive electrical waveform information, such as heartbeat information. An analog-to-digital converter (ADC) 208 digitizes the analog sensor information in a conventional manner.

The electrode 200 includes a battery 210 to power a radio module 212 to which the transmit module 202 is selectively attachable. In general, if the transmit module 202 is not present, the radio module 210 is not enabled. In one embodiment, the radio module 210 is not enabled in that no power is drawn from the battery if the transmit module is not present. In one particular embodiment, a photovoltaic device 211 is coupled to the battery 210.

In one embodiment, no power is drawn from the battery 208 by the wireless electrode unless the transmit module 212 is present. In this arrangement, a physical connection is made by mechanical manipulation of the transmit module 212. In an alternative embodiment, no power is drawn unless a receive module is present.

It is understood that radio module refers to a module that may or may not include a transmitter and/or receiver at any given time. For example, during operation of the wireless electrode, the radio module 212 includes the transmitter 202 and a receiver (if the electrode is to receive information). As described above, the device may not be active unless the radio module contains the transmit and/or receive module.

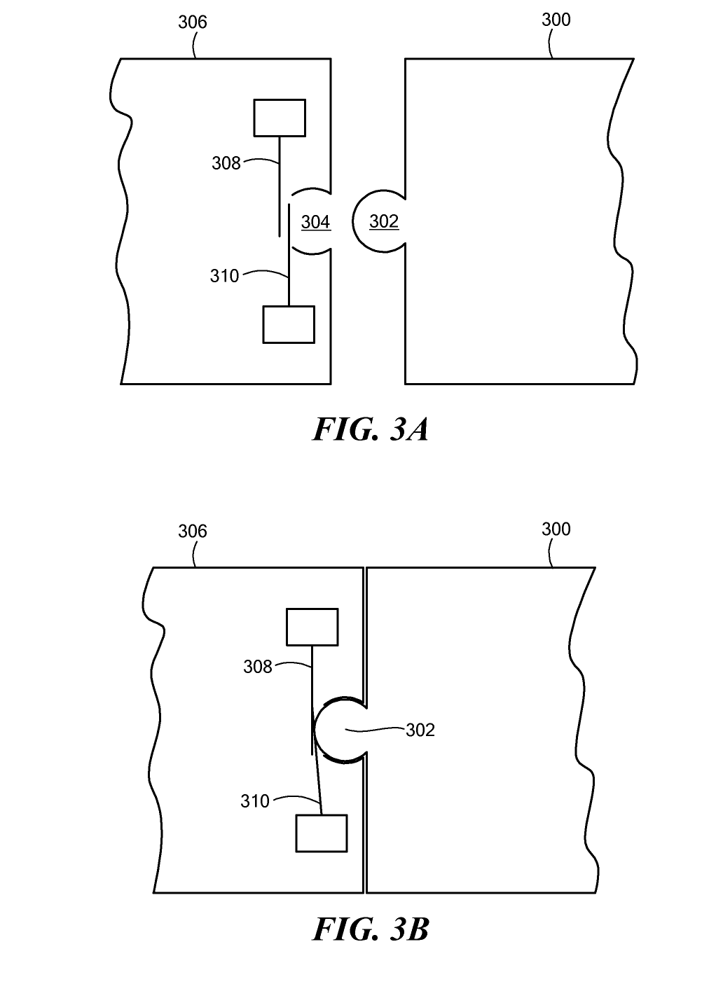

In one particular embodiment shown in FIGS. 3A and 3B, a transmit module 300 includes an activation mechanism (214 in FIG. 2) having a protrusion 302 shaped for an interference fit, e.g., snap-fit, into a cavity 304 in a radio module 306 having a shape complementary to the protrusion. The protrusion 302 can be pressed into the cavity 304. Once inserted, the protrusion 302 presses a first contact 310 into electrical contact with a second contact 308, as shown in FIG. 3B. The first and second contacts 308, 310 complete a circuit to indicate that the transmit module 300 is present for allowing the transmit module 300 to draw power from a battery and to transmit sensor information.

It is understood that a wide variety of suitable mechanical, electromechanical, optical, and other types of mechanisms can be used to detect the presence of the transmit module. The mechanisms can detect a structure, presence, and/or material to determine whether the device should be activated.

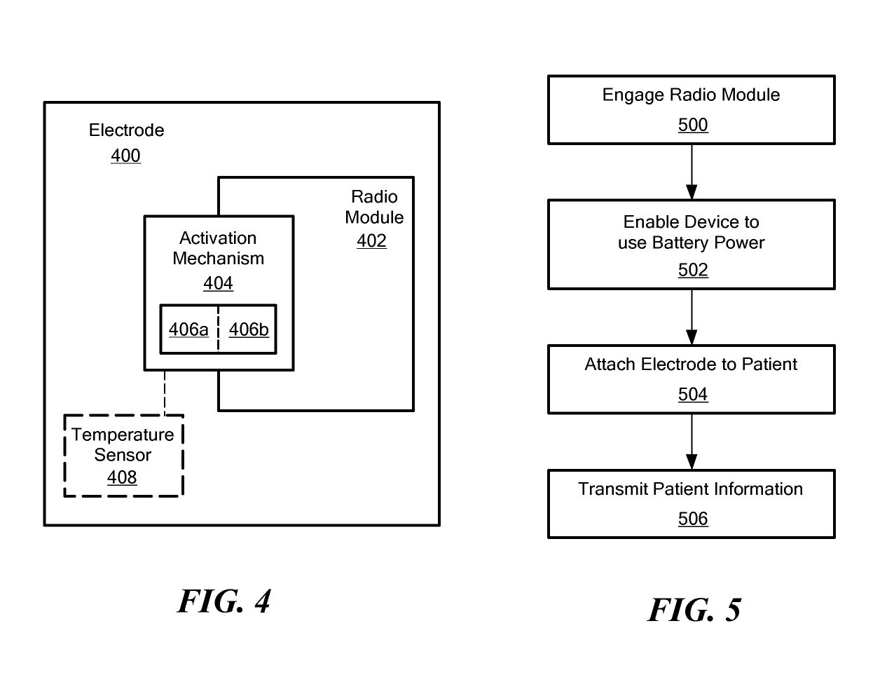

FIG. 4 shows an exemplary wireless electrode 400 coupled to a radio module 402. An activation mechanism 404 can detect the presence of the radio module 402, or components of the radio module, and activate the electrode to enable transmission of patient data.

In an exemplary embodiment, the activation mechanism 404 has a first portion 406a on the electrode and second portion 406b on the radio module 402. In one embodiment, the first portion 406a of the activation mechanism 404 includes an optical detector and the second portion 406b of the activation mechanism includes a low power light source. When the optical detector 406a detects photons from the light source after engaging the radio module 402 (or component) with the electrode 400, the activation mechanism 404 enables operation of the device, such as by closing a circuit to the battery.

In an alternative embodiment, the first portion 406a of the activation mechanism includes a magnet and the second portion 406b includes a ferrous structure. When the radio module 402 is engaged with the electrode 400, the magnet urges the ferrous structure to close a circuit which causes the activation mechanism to enable the battery to power the device.

In a further embodiment, the first portion 406a of the activation mechanism includes a proximity sensor, such as a Hall effect device, and the second portion 406b includes a ferrous portion. When the radio module 402 is engaged with the electrode 400, the Hall effect device 406a detects the ferrous portion 406b and the activation mechanism enables the battery to power the device.

In a further alternative embodiment, the first portion 406a of the activation mechanism includes an ultrasound device and the second portion 406b is at least partly formed from a material that is effective to reflect sound energy. When the radio module 402 is engaged with the electrode 400, the ultrasound device 406a detects sound energy reflected from the sound reflective material of the second portion after which the activation mechanism enables the battery to power the device. Similarly, an infra red device could be used instead of a sound device and a light reflective material can form at least part of the second portion of the activation mechanism.

The electrode 400 can further include an optional temperature sensor 408. In one embodiment, the radio module 402 is disabled when the temperature sensor 408 does not sense a temperature greater than a threshold. The electrode can transmit temperature data if desired.

In another embodiment, a wireless electrode includes an integrated radio and transmit module, i.e., the transmit module is not detachable. The wireless electrode requires the manual insertion of a pin, snap, or other structure into the radio module to enable the radio module to draw power from the battery.

FIG. 5 shows an exemplary sequence of steps for selective activation of a wireless electrode. In step 500, a radio module/component is engaged with the electrode, such as by a nurse. After engagement, an activation mechanism enables operation of the device in step 502. That is, the electrode can transmit and optionally receive information. By preventing operation of the device until the complete radio module is coupled to the electrode, battery power is not used until the device is ready to be used for a patient. In step 504, the electrode is attached to the patient and in step 506, the device transmits patient information, such as ECG signals. It is understood that the radio module can be coupled to the electrode before or after the electrode is placed on the patient's skin.

While the term transmit module is used herein, it is understood that the term “transmit module” requires transmit functionality and can further include receive functionality. That is, the wireless electrode can be transmit only, or transmit and receive.

In addition, transmit or transmit/receive modules tend to be a relatively expensive component of the electrode. With this arrangement, the transmit/radio module can be re-used. That is, the once an electrode is removed from a patient for example, the transmit module can be removed from the electrode and saved until needed for an electrode on a new patient.

Having described exemplary embodiments of the invention, it will now become apparent to one of ordinary skill in the art that other embodiments incorporating their concepts may also be used. The embodiments contained herein should not be limited to disclosed embodiments but rather should be limited only by the spirit and scope of the appended claims. All publications and references cited herein are expressly incorporated herein by reference in their entirety.

What is claimed is:

1. A wireless electrode, comprising:
an interface surface to contact a patient;
a sensor coupled to the interface surface to detect patient cardiac information;
a radio module coupled to the sensor to wirelessly transmit the cardiac information;
an energy source to power the radio module; and
an activation mechanism coupled to the energy source, the activation mechanism having an activated state in which power from the energy source is delivered to the radio module and a non-activated state in which power from the energy source is not delivered to the radio module,
wherein the radio module comprises a transmitter insertable into the electrode and wherein insertion of the transmitter into the electrode transitions the activation mechanism to the activated state.

2. (canceled)

3. The electrode according to claim 1, wherein the activation mechanism comprises a deformable receptacle to detect insertion of the transmitter into the electrode for transition to the activated state.

4-5. (canceled)

6. The electrode according to claim 1, further including a temperature sensor.

7. The electrode according to claim 6, wherein the radio module is disabled when the temperature sensor does not sense a temperature greater than a threshold.

8. The electrode according to claim 6, wherein the radio module transmits information from the temperature sensor.

9. The electrode according to claim 1, wherein the energy source comprises a battery.

10. The electrode according to claim 9, wherein the energy source further comprises a photovoltaic device coupled to the battery.

11. The electrode according to claim 1, wherein the electrode comprises one or more of an ECG electrode, a blood pressure electrode and/or temperature electrode.

12. The electrode according to claim 1, wherein the activation mechanism comprises one or more of an optical detector, a magnetic detector, a proximity sensor, an ultrasound sensor, and/or an infrared sensor.

13. A system to obtain patient information, comprising:
a plurality of wireless electrodes, each comprising:
an interface surface to contact a patient;
a sensor coupled to the interface surface to detect patient cardiac information;
a radio module coupled to the sensor to wirelessly transmit the cardiac information;
an energy source to power the radio module; and
an activation mechanism coupled to the energy source, the activation mechanism having an activated state in which power from the energy source is delivered to the radio module and a non-activated state in which power from the energy source is not delivered to the radio module; and
a transmit/receive module to receive the cardiac information from the radio module; and
a monitor in communication with the transmit/receive module to display the cardiac information and generate alerts based upon the cardiac information,
wherein the radio module comprises a transmitter insertable into the electrode and wherein insertion of the transmitter into the electrode transitions the activation mechanism to the activated state.

14. The system according to claim 13, wherein at least one of the wireless electrodes includes a temperature sensor.

15. The system according to claim 13, wherein the radio module is disabled when the temperature sensor does not sense a temperature greater than a threshold.

16. The system according to claim 13, wherein the energy source comprises a photovoltaic mechanism coupled to a battery.

17. The system according to claim 13, wherein the activation mechanism cannot transition to the activated state unless a transmitter is present in the radio module.

18. A method of providing a wireless electrode, comprising:
providing an interface surface to contact a patient;
providing a sensor coupled to the interface surface to detect patient cardiac information;
providing a radio module coupled to the sensor, the radio module including a transmitter to wirelessly transmit the cardiac information;
providing an energy source to power the radio module; and
providing an activation mechanism coupled to the energy source, the activation mechanism having an activated state in which power from the energy source is delivered to the radio module and a non-activated state in which power from the energy source is not delivered to the radio module,
wherein the radio module comprises a transmitter insertable into the electrode and wherein insertion of the transmitter into the electrode transitions the activation mechanism to the activated state.

19. The method according to claim 18, further including providing a temperature sensor, wherein the radio module is disabled when the temperature sensor does not sense a temperature greater than a threshold.

20. (canceled)

21. A method, comprising:
applying a wireless electrode to a patient to obtain patient cardiac information;
manipulating an activation mechanism of the wireless electrode from a non-activated state in which power from an energy source is not delivered to a radio module to an activated state in which power from the energy source is delivered to the radio module; and
monitoring the cardiac information transmitted by the radio module,
wherein the radio module comprises a transmitter insertable into the electrode and wherein insertion of the transmitter into the electrode transitions the activation mechanism to the activated state.

22. The electrode according to claim 1, wherein the activation mechanism comprises an optical detector to detect insertion of the transmitter into the electrode for transition to the activated state.

23. The electrode according to claim 1, wherein the activation mechanism comprises a magnetic sensing element to detect insertion of the transmitter into the electrode for transition to the activated state.

24. The electrode according to claim 1, wherein the activation mechanism comprises an ultrasound device to detect insertion of the transmitter into the electrode for transition to the activated state.

25. A wireless electrode, comprising:
an interface surface to contact a patient;
a sensor coupled to the interface surface to detect patient cardiac information;
a radio module coupled to the sensor to wirelessly transmit the cardiac information;
an energy source to power the radio module; and
a temperature sensor,
wherein generally no power from the energy source is delivered to the radio module when the temperature sensor does not sense a temperature greater than a threshold.
